(19) United States
(12) Patent Application Publication
(10) Pub. No.: US 2026/0224326 A1
(43) Pub. Date: Aug. 6, 2026

(54) SPRING ARM DEVICE AND SUPPORT ARM SYSTEM FOR HOLDING A MEDICAL APPLIANCE

(71) Applicant: Ondal Medical Systems GmbH, Hünfeld (DE)

(72) Inventor: Georg MURATIDI, Oberaula (DE)

(21) Appl. No.: 19/148,439

(22) PCT Filed: Jan. 16, 2024
(86) PCT No.: PCT/EP2024/050922

§ 371 Date: Aug. 4, 2025

(30) Foreign Application Priority Data
Jan. 17, 2023 (EP) 23152046.1

(51) Int. Cl. A61B 90/50

(52) U.S. Cl. CPC A61B 90/50; A61B 2090/508

(57) ABSTRACT

The present invention relates to a spring arm device (1) for a support arm system (100) for holding a medical appliance, comprising a spring tube (3) and a spring rod (4) arranged at least partially in the interior of the spring tube (3) and movable relative to the spring tube (3) in the longitudinal direction (2) of the spring tube (3). The spring arm device is further characterized by a clamping unit (30). For releasably applying a clamping force to the spring rod (4), the clamping unit is arranged on the spring tube (3) in a fixed position as viewed in the longitudinal direction (2). Also disclosed is a support arm system (100) for holding at least one medical appliance, comprising at least one spring arm device (1).

### TECHNICAL FIELD

The present invention relates to a spring arm device for a support arm system for holding a medical appliance and a support arm system for holding a medical appliance.

### PRIOR ART

Support arm systems are used, for example, in operating rooms to hold a medical appliance or piece of equipment in a locally relocatable manner. For example, a support arm can be movably anchored to a ceiling, wall or floor by means of a bearing arrangement. It is also possible to mount it on a frame with wheels. A support arm system designed to hold medical appliances can also be referred to as a medical support system. Support arm systems usually have at least one axis of movement so that the held medical appliance, which is also referred to as a medical technical appliance and can have, for example, an operating lamp, a monitor, examination devices, one or more injectors, surgical instruments or dental drills, can be moved according to the respective requirements. The bearing that enables the movement can also be referred to as an articulation device or support arm articulation device, wherein this can also comprise a support arm. This allows the medical appliance to be placed in a comfortable and safe position for performing the respective medical procedures and monitoring. The local relocation of medical appliances is essential for many medical procedures in order to facilitate the work of medical staff or to enable it at all.

However, by pivoting a support arm, a lever arm can be changed with regard to its bearing. In particular, when the support arm is pivoted about a substantially horizontal axis, whereby the height of the medical appliance held thereon can be changed, a lever arm of the weight force of the medical appliance is changed relative to the fastening or mounting of the support arm. Nevertheless, it is desired that the medical appliance remains in a position adjusted by pivoting the support arm and that an adjustment of the position of the medical appliance, in particular against the force of gravity, should be possible without excessive effort by an operator.

For this purpose, spring-balanced support arms, referred to here as spring arms or spring arm devices, are often used. Two arm elements are provided that can be moved relative to each other and support each other by means of a spring. By pivoting the spring arm, the lever arm of the spring force changes correspondingly to the lever arm of the supported load, whereby the medical appliance can be held equally well in different positions. Alternatively or additionally, the spring can also be compressed or stretched by pivoting, which also allows a corresponding support force to be changed.

Such support arm articulation devices or spring arm devices can be found, for example, in WO 2016/074787 A1 or EP 3 861 958 A1.

### PRESENTATION OF THE INVENTION

Based on the known prior art, it is an object of the present invention to provide an improved spring arm device for a support arm system for holding a medical appliance, as well as a corresponding support arm system for holding a medical appliance. In particular, it should be possible to easily and cost-effectively hold medical appliances permanently in a given position. The object is achieved by a device for a support arm system for holding a medical appliance having the features of claim 1. Advantageous developments will be apparent from the dependent claims, the description, and the figures.

Accordingly, a spring arm device for a support arm system for holding a medical appliance is proposed, comprising a spring tube and a spring rod arranged at least partially in the interior of the spring tube and movable relative to the spring tube in the longitudinal direction of the spring tube.

The spring arm device further comprises a clamping unit for releasably applying a clamping force to the spring rod, which clamping unit is arranged on the spring tube in a fixed position as viewed in the longitudinal direction of the spring tube. By applying the clamping force via the clamping unit, a displacement of the spring rod relative to the clamping unit in the longitudinal direction and accordingly to the spring tube fixedly connected to the clamping unit can be prevented. In other words, the clamping unit can lock the spring rod relative to the spring tube. The spring rod is fixed in a fixed position relative to the spring tube by the clamping unit when the latter is in a state of application of the clamping force. This means in particular that the spring rod, when clamped, is locked relative to the spring tube against displacement along the longitudinal direction in both directions.

If the clamping unit is in the released state, in which the clamping unit does not exert any clamping force on the spring rod, the spring rod can be moved relative to the spring tube.

Due to the clamping unit, in particular, a continuous locking of the position of the spring rod relative to the spring tube can be provided. For example, in contrast to conventional devices with a predetermined number of spaced-apart fixed locking positions, the clamping force can be applied via the proposed clamping unit in essentially any position of the spring rod relative to the spring tube and thus the spring arm can be fixed in essentially any position. The continuous locking is provided at least over a predetermined range of movement of the spring tube relative to the spring tube in the longitudinal direction of the spring tube, in other words in the direction of the longitudinal axis of the spring tube.

Accordingly, the present spring arm device enables a precise and secure holding of a spring arm of a support arm system in a fixed position, wherein the spring arm device simultaneously has a comparatively simple structure.

The possibility of fixing (i.e. locking) the spring arm device in a predetermined fixed position can be particularly advantageous in applications in which the load on the support arm system comprising the spring arm device changes.

For example, when holding a medical injector, the suspended load held by a spring arm or spring arm device may be reduced over time due to the emptying of the liquid from the injector. Due to this reduction, it may happen that the preload acting on the spring arm of a preload element of the spring arm device and the supported load are no longer in balance. As a result, the spring arm could move from its intended position and thereby move the injector upwards. The proposed clamping unit allows the position of the spring arm or spring arm device to be fixed so that the injector is always held in the intended position and a patient can be treated safely.

Analogous to the above, in the case of medical appliances designed as documentation terminals to which a monitor with a keyboard is connected, a temporary increase in the supported load may occur if, when operating the keyboard, an additional operating force, caused by typing on the keyboard, is introduced into the support arm system. By means of the solution according to the invention, the spring arm device can be locked in a predetermined fixed position and prevented from lowering due to the acting operating force.

Furthermore, by locking the spring arm device by applying the clamping force through the clamping unit, it is possible to reduce or even completely prevent any migration or drifting of the spring arm device, or more precisely, a vibration-induced continuous or discontinuous displacement of the spring rod relative to the spring tube, due to vibrations occurring during operation of the medical appliance held by the support arm system.

The basic structure of a spring arm device can also be found in WO 2016/074787 A1, in particular page 14, line 14 to page 20, line 20, and EP 3 861 958 A1, in particular paragraphs 48 to 59, which are incorporated herein by reference.

The clamping unit is preferably designed such that by applying the clamping force to the spring rod, the spring rod is locked in its position relative to the spring tube.

The clamping unit preferably has a contact surface for applying the clamping force to an outer peripheral portion of the spring rod, wherein the clamping unit is preferably designed such that, in the state of application of the clamping force, the contact surface is pressed against the outer peripheral portion with a predetermined pressing force. This allows for particularly secure clamping of specified height. By providing the flat pressure via the contact surface, the required clamping force can be reproducibly transferred to the spring rod without causing excessive local pressure on the spring rod, which could otherwise damage the material of the spring rod. The clamping force can therefore be transmitted longitudinally distributed over the length of the contact surface. The contact surface may be a substantially cylindrical surface the longitudinal axis of which extends in the longitudinal direction.

According to an embodiment, the clamping unit can comprise at least one clamping piece, preferably at least one clamping jaw, and/or a clamping ring, wherein the at least one clamping piece and/or the clamping ring preferably comprises the contact surface.

The clamping ring preferably comprises an annular base body with at least one radially extending slot. In other words, the clamping ring is a ring segment with opposite free ends that limit the width of the slot in the circumferential direction of the ring segment. The opposite free ends are preferably spaced apart from each other by a predetermined distance in an unloaded state.

The radial inner surface of the clamping ring represents the contact surface. If the free ends of the clamping ring are pressed towards each other from the unclamped state, the pitch circle diameter on which the radial inner surface is located is reduced accordingly, so that the spring rod guided by the clamping ring can be clamped.

Preferably, the clamping unit comprises a manually operable actuating unit for applying and releasing the clamping force. This preferably comprises a clamping lever or an eccentric lever. This allows a person using or operating the spring arm device to easily manually adjust and release the locking of the movement of the spring rod and thus of the spring arm device.

Alternatively or additionally, the actuating unit may comprise a manually operable handle, preferably a lever handle, a screw handle or a star handle.

According to an optional embodiment, the clamping unit is held stationary relative to the spring tube by the actuating unit. In other words, the actuating unit can be designed to engage with the spring tube and the spring rod in such a way that the clamping unit is prevented from being displaced in the longitudinal direction relative to the spring tube. This eliminates the need for a separate attachment of the clamping unit to the spring tube, for example by screwing the clamping unit to the spring tube. In other words, the actuating unit is held stationary on the spring tube and the actuating unit holds the clamping unit in the specified position with respect to the longitudinal direction. Forces in the longitudinal direction are therefore transmitted from the clamping unit via the actuating unit to the spring tube. The actuating unit therefore also takes on the function of a fastening device for the clamping unit. According to this optional embodiment, it can be provided that the actuating unit, or a component of the actuating unit, is supported in a receptacle provided on the spring tube, for example in the form of a receiving bore. The receiving bore can extend transversely to the longitudinal direction, for example skewed, i.e. without intersecting the center axis of the spring tube and/or the center axis of the spring rod. The clamping unit is positioned relative to the spring tube by the actuating unit, i.e. it is held stationary on the spring tube.

For example, the actuating unit may comprise or be designed as a clamping lever comprising a threaded bolt and a handle, such as a lever handle, attached to one end thereof. At the other end, the threaded bolt can be screwed into a thread formed in the clamping unit, for example in a clamping ring of the clamping unit.

The threaded bolt can also be guided through the spring tube in a receiving bore provided in the spring tube. This allows the threaded bolt to be fixed in its position relative to the spring tube and to be held displaceable in the receiving bore only in the axial direction and to be rotatable in it about the axial direction of the receiving bore. Since the threaded bolt is also screwed to the clamping unit, the clamping unit is then held stationary relative to the spring tube in the longitudinal direction.

According to an optional embodiment, the spring rod comprises a threaded rod which comprises a thread at least in one portion. The spring rod, preferably its threaded rod, can have a cylindrical surface portion providing the outer peripheral portion. Alternatively or additionally, the spring rod may also comprise a sleeve, preferably screwed onto a thread of the threaded rod, which provides the outer peripheral portion. Preferably, the sleeve is arranged in a rotationally fixed manner on the spring rod, preferably on the threaded rod, wherein the sleeve is preferably fixed in its position relative to the spring rod, preferably to the threaded rod, by means of a securing element, preferably a securing ring, a securing pin or a securing screw. Alternatively or additionally, the sleeve can be pressed onto the spring rod or the spring rod, preferably the threaded rod. Alternatively or additionally, the threaded rod and the sleeve can be screwed together in a clamped state. Furthermore, alternatively or additionally, the screw connection between the threaded rod and the sleeve can be designed to be self-locking.

The outer peripheral portion may further be a portion of a thread of the threaded rod.

The outer peripheral portion is advantageously designed to correspond to the contact surface of the clamping unit.

The clamping unit, advantageously comprising a clamping ring, can be changed, for example, via the actuating unit between a released state and a state in which the clamping force is applied. In the released state, the outer peripheral portion can slide relative to the contact surface and thus the spring rod can be moved relative to the spring tube. This can be made possible, for example, by an inner radius of the clamping unit, advantageously of the clamping ring, defined by the contact surface, in the released state, being larger than an outer radius of the outer peripheral portion.

The outer peripheral portion advantageously has a predetermined length in the longitudinal direction. The spring rod can be moved relative to the clamping unit along the length of the outer peripheral portion and the clamping force can be applied continuously by the clamping unit at any point on the outer peripheral portion.

The clamping unit is advantageously arranged circumferentially around the spring rod and/or radially outwardly around the spring rod with respect to the longitudinal direction. In other words, the clamping unit or parts thereof do not penetrate the spring rod in a direction transverse to the longitudinal direction, which would otherwise weaken the load-bearing capacity of the spring rod and also increase the manufacturing effort.

The spring rod is advantageously designed to be continuous in the region of the clamping unit and/or in the region of the outer peripheral portion.

The threaded rod is advantageously formed in one piece and/or is continuous. In other words, the threaded rod is advantageously not multi-part and segmented. Accordingly, no connections between the segments need to be provided.

The spring rod is preferably mounted translationally in the spring tube, preferably slidingly mounted, wherein the spring rod preferably comprises at least one slide which is mounted translationally relative to the spring tube, preferably slidingly mounted. Preferably, the spring rod comprises on a first side a first slide mounted translationally relative to the spring tube and on a second side a second slide mounted translationally relative to the spring tube.

According to a preferred, i.e. optional, embodiment, the spring tube is arranged on a first side on a first fastening unit so as to be rotatable about a first axis of rotation, and the spring rod is connected to a lever element on the first side so as to be rotatable about a second axis of rotation preferably oriented parallel to the first axis of rotation. The lever element is preferably arranged on its side opposite the spring rod-spaced from the first axis of rotation-on the first fastening unit so as to be rotatable about a third axis of rotation which is preferably oriented parallel to the first axis of rotation. Accordingly, a spring arm gear can be provided.

In order to apply a preload to the spring rod, a spring element, preferably a compression spring, can be provided in the spring tube for preloading the spring rod relative to the spring tube, wherein the spring element is preferably supported at one end on the spring tube, preferably on a shoulder provided in the spring tube, preferably comprising an recess on the spring tube, and is supported at another end on the spring rod, preferably on a shoulder provided on the spring rod.

According to a preferred embodiment, the contact surface and/or the outer peripheral portion at least partially comprise a coating, preferably a brake pad layer, which increases the coefficient of friction between the contact surface and the outer peripheral portion compared to a material pairing of the base materials of the part comprising the contact surface and the part comprising the outer peripheral portion.

The above-mentioned object is further achieved by a support arm system for holding at least one medical appliance having the features of claim 11. Advantageous further developments emerge from the present description and the figures.

Accordingly, a support arm system for holding at least one medical appliance is proposed, which comprises at least one spring arm device according to any one of the preceding embodiments.

The advantages and effects described with regard to the spring arm device are also achieved by the support arm system, which is why they will not be described again here in order to avoid redundancies.

### BRIEF DESCRIPTION OF THE FIGURES

Preferred further embodiments of the invention are explained in more detail by the following description of the figures. In particular:

FIG. 1 schematically shows a support arm system with a spring arm device;

FIG. 2 schematically shows a perspective side view of a partial region on a second side of the spring arm device from FIG. 1;

FIG. 3 schematically shows a perspective sectional view through the spring arm device of FIGS. 1 and 2 at the level of a clamping unit transverse to a longitudinal direction; and

FIG. 4 schematically shows a perspective sectional view in the longitudinal direction through a partial region of the spring arm device from FIGS. 1 to 3.

### DETAILED DESCRIPTION OF PREFERRED EMBODIMENTS

Preferred embodiments are described below with reference to the figures. In this case, identical, similar or equivalent elements in the different figures are provided with identical reference numerals, and a repeated description of these elements is partly omitted in order to avoid redundancies.

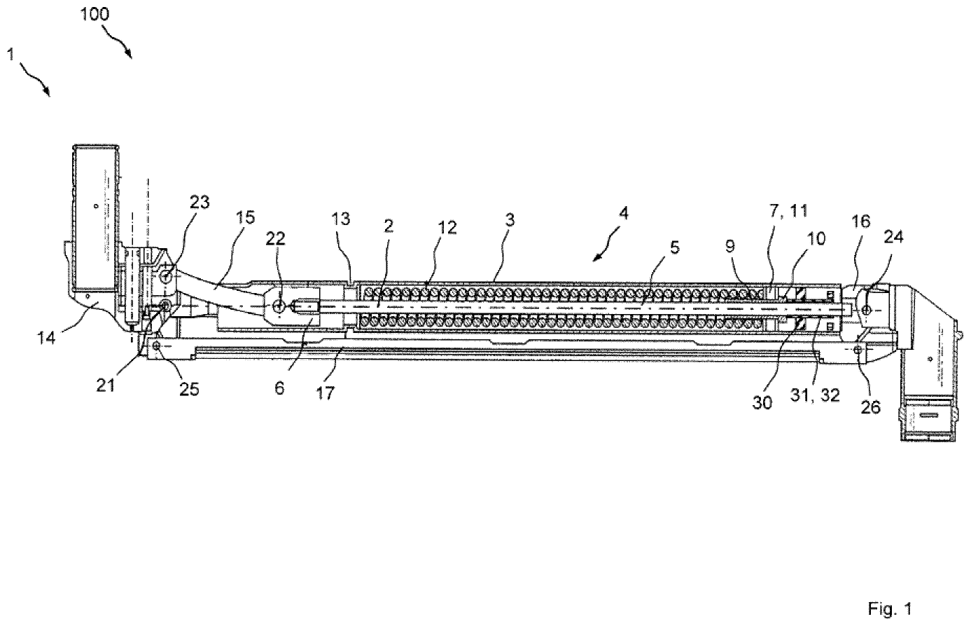

FIG. 1 schematically shows a support arm system 100 with a spring arm device 1. The support arm system 100 is designed to hold a medical appliance (not shown).

The spring arm device 1 comprises a spring tube 3 extending in a longitudinal direction 2. A spring rod 4 is arranged inside the spring tube 3 and is displaceable in the longitudinal direction 2 relative to the spring tube 3.

The spring rod 4 has a threaded rod 5 extending in the longitudinal direction 2, which is fastened, in this case screwed, on a first side to a first slide 6 mounted in a translationally sliding manner relative to the spring tube 3, and is connected on a second side to a second slide 7 mounted in a translationally sliding manner relative to the spring tube 3. In the present case, the second slide comprises a threaded bushing 9 with a shoulder 10, onto which a sliding ring 11 is pushed.

The sliding ring 11 is also designed as a stop on the second side for a spring element 12, in this case designed as a compression spring, for preloading the spring rod 4 relative to the spring tube 3. The spring element 12 is supported on the first side on a recess 13 in the spring tube 3. The distance in the longitudinal direction 2 between the sliding ring 11 serving as the first stop on the second side and the recess 13 serving as the second stop on the first side is smaller than a zero length of the spring element 12, which it has in a relaxed state. The spring element 12 is therefore preloaded. The amount of preload of the spring element 12 can be changed by changing the position of the second stop relative to the threaded rod 5 in the longitudinal direction 2. For this purpose, the threaded bushing 9 can be rotated relative to the threaded rod 5. According to the present, i.e. optional, embodiment of the spring arm device 1, the screw connection between the threaded bushing 9 and the threaded rod 5 is designed to be self-locking. Alternatively, the threaded bushing can also be secured in its position relative to the threaded rod 5 by means of an optional securing element (not shown).

The spring tube 3 is arranged on the first side on a first fastening unit 14 so as to be rotatable about a first axis of rotation 21. Furthermore, the spring rod 4 is connected to the first fastening unit 14 via a lever element 15. More precisely, the spring rod 4 is connected to the lever element 15 on the first side so as to be rotatable about a second axis of rotation 22 oriented parallel to the first axis of rotation 21, and the lever element 15 is arranged on its side opposite the spring rod 4 so as to be rotatable about a third axis of rotation 23 spaced from the first axis of rotation 21 and oriented parallel to the first axis of rotation 21 on the first fastening unit 14.

Accordingly, the preloaded spring element 12 provides a preload of the spring tube 3, wherein the preload counteracts a weight force acting on the spring tube 3.

On the second side, the spring tube 3 is rotatably arranged on a second fastening unit 16 via a fourth rotation axis 24 oriented parallel to the first rotation axis 21.

The spring arm device 1 further comprises a pull-push rod 17, which is rotatably arranged on the first fastening unit 14 via a fifth rotational axis 25 oriented parallel to the first rotational axis 21 and rotatably arranged on the second fastening unit 16 via a sixth rotational axis 26 oriented parallel to the fourth rotational axis 24.

Accordingly, the spring arm device forms an articulation device which provides parallel guidance of the second fastening unit 16 relative to the first fastening unit 14.

A medical appliance (not shown) or a further spring arm device can be attached to the second fastening unit 16, wherein the second fastening unit 16 then represents the first fastening unit of this further spring arm device.

Because the lever element 15 and the spring tube 3 are mounted at a distance from one another on the first fastening element or unit 14, a different lever arm or angle results depending on the angular position of the spring tube 3 about the first axis of rotation 21, by means of which the lever element 15 supports the spring tube 3 via the spring rod 4 and the spring element 12. Thus, in different positions of the spring arm device 1 or the spring tube 3, a similar or even essentially the same weight force can be supported equally.

The combination of spring tube 3, spring arm 4 and spring element 12 can be understood as a balanced spring arm.

Further details on the design of the spring arm device or the spring arm can be found in WO 2016/074787 A1, in particular page 14, line 14 to page 20, line 20, and EP 3 861 958 A1, in particular paragraphs 48 to 59, to the content of which reference is made again at this point.

The spring arm device 1 further comprises a clamping unit 30 for releasably applying a clamping force to the spring rod 4. According to this optional embodiment, the clamping unit 30 is arranged in a fixed position on the spring tube 3 in the region of the second side as viewed in the longitudinal direction 2.

The spring rod 4 comprises a cylindrical sleeve 31 screwed onto the threaded rod 5, which provides an outer peripheral portion 32.

According to this optional embodiment, the sleeve 31 is arranged in a rotationally fixed manner on the threaded rod 5 via self-locking. Alternatively or additionally, the sleeve 31 can also be fixed in its position on the threaded rod 5 by means of a securing element (not shown), such as a securing ring, a securing pin or a securing screw. The sleeve 31 can also be pressed onto the threaded rod 5 and/or the threaded rod 5 and the sleeve 31 can be screwed together in a clamped state, for example by the sleeve 31 being counter-tightened against the threaded bushing 9.

The clamping unit 30 is designed such that by applying the clamping force to the spring rod 4, the spring rod 4 is locked in its position relative to the spring tube 3.

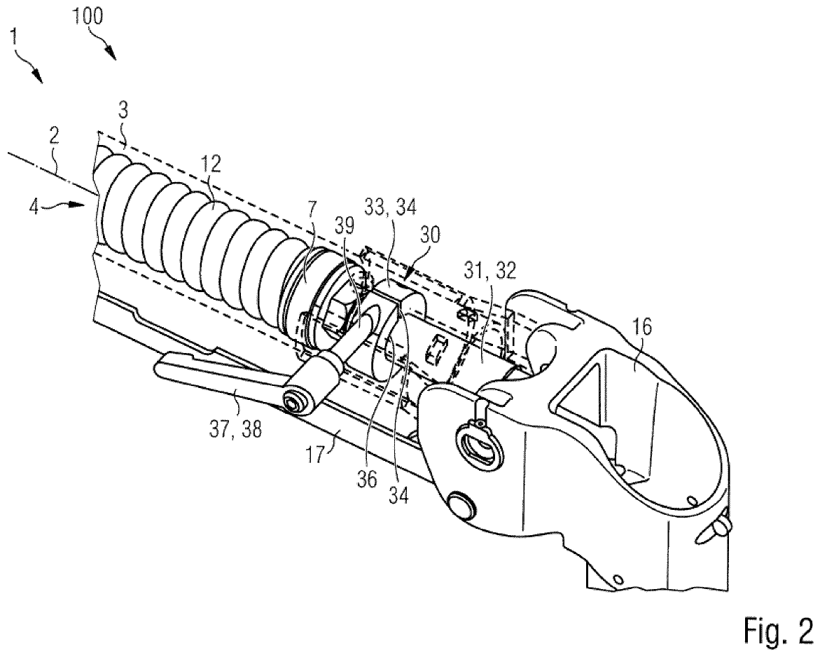

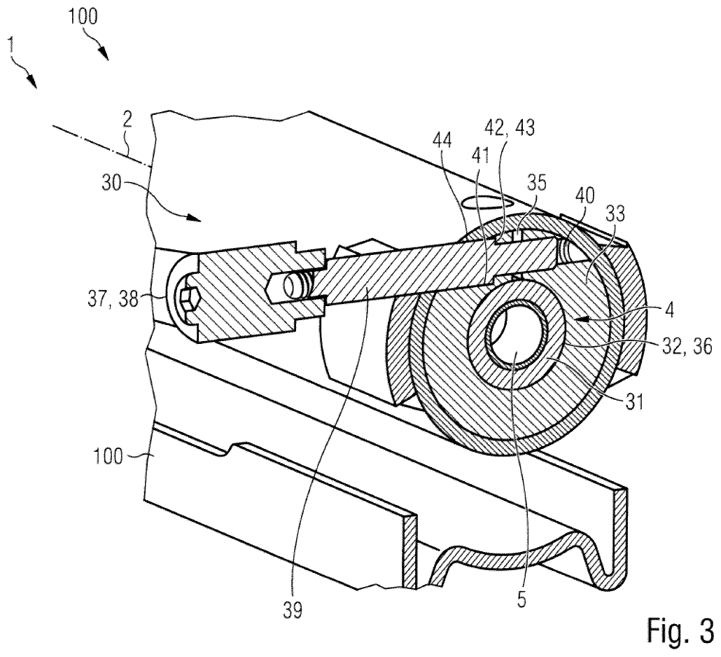

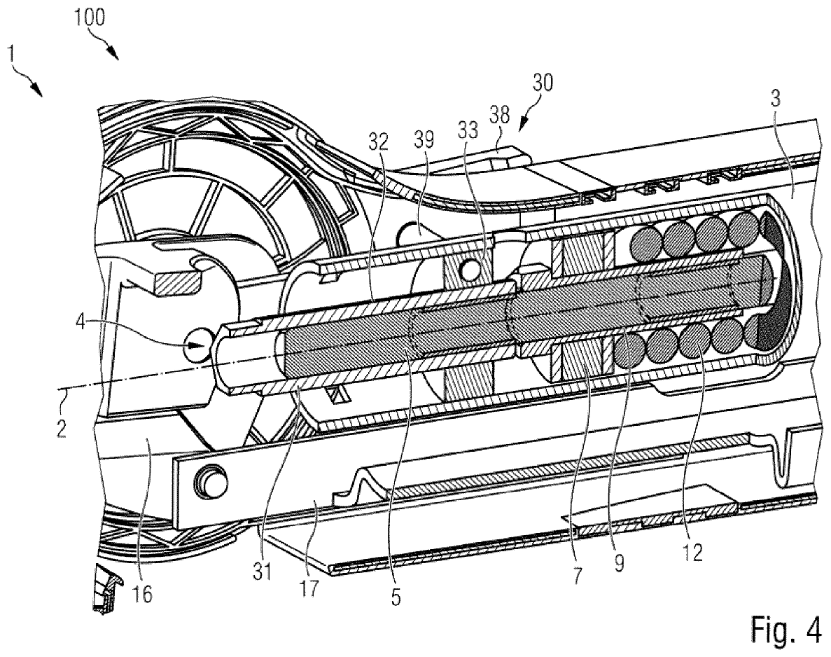

Advantageous details of the clamping unit 30 are described in more detail below with reference to FIGS. 2 to 4. FIG. 2 schematically shows a perspective side view of a partial region on the second side of the spring arm device 1 from FIG. 1, wherein the spring tube 3 is shown partially transparent in order to be able to recognize the components accommodated therein. FIG. 3 shows a schematic perspective sectional view through the spring arm device 1 from FIGS. 1 and 2 at the level of the clamping unit 30. FIG. 4 schematically shows a perspective sectional view in the longitudinal direction 2 through a partial region of the spring arm device 1 from FIGS. 1 to 3.

In the present case, the clamping unit 30 comprises a clamping ring 33 which is inserted in the interior of the spring tube 3. The clamping ring 33 is formed in the present case from an annular base body 34 with a slot 35 extending radially through the base body 34. The base body 34 is thus a ring segment with free ends opposite each other at the slot 35, wherein the opposite free ends are spaced apart from each other by a predetermined distance (the width of the slot 35) in an unloaded state of the clamping ring 33.

If the free ends of the clamping ring 33 are pressed towards each other from the unclamped state, the pitch circle diameter on which the radial inner surface is located decreases accordingly. The radial inner surface of the clamping ring 33 represents a contact surface 36 for applying the clamping force to the outer peripheral portion 32 of the spring rod 4. When the clamping ring 33 is compressed, the spring rod 4 guided through the clamping ring 33 can be clamped.

The clamping ring 33 can be transferred between a released state and a state applying the clamping force via an actuating unit designed in the present case in the form of a clamping lever 37 (see FIGS. 2 and 3). In the released state, the outer peripheral portion 32 can slide relative to the contact surface 36 and thus the spring rod 4 can be moved relative to the spring tube 3. In the present case, in the released state, the inner radius of the clamping ring 33 defined by the contact surface 36 is larger than the outer radius of the outer peripheral portion 32.

The clamping lever 37 in the present case comprises a threaded bolt 39 and a lever handle 38 attached to one end thereof. At the other end, the threaded bolt 39 is screwed into a thread 40 formed in the clamping ring 33. Opposite the gap or slot 35, the threaded bolt 39 is guided in a bore 41 in the clamping ring 33 and is supported by a bolt shoulder 42 on a shoulder 43 in the bore 41 in the axial direction of the threaded bolt 39.

The threaded bolt 39 is further guided through the spring tube 3 in a receiving bore 44 provided in the spring tube 3. This allows the threaded bolt 39 to be fixed in its position relative to the spring tube 3 and to be held displaceable in the receiving bore 44 only in the axial direction thereof and to be rotatable in it about the axial direction of the receiving bore 44. Since the threaded bolt 39 also runs through the bore 41, the clamping ring 33 and thus the clamping unit 30 are also held stationary with respect to the longitudinal direction 2 relative to the spring tube 3.

By screwing the threaded bolt 39 deeper into the thread 40 via the clamping lever 37, the pitch circle diameter of the contact surface 36 is reduced so that the clamping force can be applied to the spring rod 4, more precisely to the sleeve 31. The clamping force can be released again by unscrewing the threaded bolt 39 via the clamping lever 37.

By adjusting the clamping lever 37 to a predetermined angular position about the longitudinal axis of the threaded bolt 39, which can optionally be indicated by a marking on the outside of a housing part of the spring tube device 1 not shown here, the contact pressure between the contact surface 36 and the outer peripheral portion 32 can have a predetermined level.

The contact surface 36 and/or the outer peripheral portion 32 may optionally have at least partially a coating, preferably a brake pad layer, which preferably increases the coefficient of friction between the contact surface 36 and the outer peripheral portion 32.

The maximum range of movement of the spring rod 4 relative to the spring tube 3, at which the clamping unit 30 can exert the clamping force on the spring rod 4, is predetermined by the length in the longitudinal direction 2 of the outer peripheral portion 32, which can be seen in FIG. 4.

Where applicable, all individual features shown in the embodiments may be combined and/or exchanged with one another without departing from the scope of the invention.

### LIST OF REFERENCE NUMERALS

1 spring arm device
2 longitudinal direction
3 spring tube
4 spring rod
5 threaded rod
6 first sled
7 second sled
9 threaded bushing
10 shoulder
11 sliding ring
12 spring element
13 recess
14 first fastening unit
15 lever element
16 second fastening unit
17 push-pull rod
21 first axis of rotation
22 second axis of rotation
23 third axis of rotation
24 fourth axis of rotation
25 fifth axis of rotation
26 sixth axis of rotation
30 clamping unit
31 sleeve
32 outer peripheral portion
33 clamping ring
34 base body
35 slot
36 contact surface
37 clamping lever
38 lever handle
39 threaded bolt
40 thread
41 bore
42 bolt shoulder
43 shoulder
44 receiving bore
100 support arm system

1. A spring arm device for a support arm system for holding a medical appliance, comprising:
a spring tube and a spring rod arranged at least partially in the interior of the spring tube and movable relative to the spring tube in the longitudinal direction of the spring tube; and
a clamping unit for releasably applying a clamping force to the spring rod is arranged in a fixed position on the spring tube as viewed in the longitudinal direction.

2. The spring arm device according to claim 1, wherein the clamping unit is designed such that by applying the clamping force to the spring rod, the spring rod is locked in its position relative to the spring tube.

3. The spring arm device according to claim 1, wherein the clamping unit has a contact surface for applying the clamping force to an outer peripheral portion of the spring rod.

4. The spring arm device according to claim 3, wherein the clamping unit is designed such that, in the state in which the clamping force is applied, the contact surface is pressed against the outer peripheral portion with a predetermined pressing force.

5. The spring arm device according to claim 1, wherein the clamping unit comprises at least one clamping piece, preferably at least one clamping jaw, and/or a clamping ring, wherein the at least one clamping piece and/or the clamping ring comprises the contact surface.

6. The spring arm device according to claim 1, wherein the clamping unit comprises an actuating unit for applying and releasing the clamping force.

7. The spring arm device according to claim 6, wherein the actuating unit comprises a clamping lever or an eccentric lever, and/or the actuating unit comprises a manually operable handle, preferably a lever handle, a screw handle or a star handle.

8. The spring arm device claim 1, wherein the spring rod comprises a threaded rod.

9. The spring arm device according to claim 8, wherein the threaded rod has a cylindrical surface portion providing the outer peripheral portion and/or comprises a sleeve screwed onto a thread of the threaded rod and providing the outer peripheral portion.

10. The spring arm device according to claim 9, wherein the sleeve is arranged in a rotationally fixed manner on the threaded rod.

11. The spring arm device according to claim 10, wherein the sleeve is fixed in its position relative to the threaded rod by means of a securing element, a securing ring, a securing pin or a securing screw on the threaded rod, and/or the sleeve is pressed onto the threaded rod, and/or the threaded rod and the sleeve are screwed together in a clamped state, and/or the screw connection of the threaded rod and the sleeve is designed to be self-locking.

12. The spring arm device according to claim 1, wherein the spring rod is mounted translationally, preferably slidingly, in the spring tube, wherein the spring rod preferably comprises at least one slide mounted translationally, preferably slidingly, relative to the spring tube, wherein the spring rod preferably comprises on a first side a first slide mounted translationally relative to the spring tube and on a second side a second slide mounted translationally relative to the spring tube.

13. The spring arm device according to claim 1, wherein the spring tube is arranged on a first fastening unit so as to be rotatable about a first axis of rotation on a first side, and the spring rod is connected to a lever element on the first side so as to be rotatable about a second axis of rotation oriented parallel to the first axis of rotation, and the lever element is arranged on its side opposite the spring rod so as to be rotatable about a third axis of rotation spaced from the first axis of rotation and oriented parallel to the first axis of rotation on the first fastening unit.

14. The spring arm device according to claim 1, wherein a spring element, preferably a compression spring, is provided in the spring tube for preloading the spring rod relative to the spring tube, wherein the spring element is preferably supported at one end on the spring tube, preferably on a shoulder provided in the spring tube, and is supported at the other end on the spring rod, preferably on a shoulder provided on the spring rod.

15. The spring arm device according to claim 1, wherein the contact surface and/or the outer peripheral portion at least partially have a coating, preferably a brake lining layer, which increases the coefficient of friction between the contact surface and the outer peripheral portion.

16. A support arm system for holding at least one medical appliance, comprising at least one spring arm device according to claim 1.
